(19) United States
(12) Patent Application Publication
(10) Pub. No.: US 2003/0198593 A1
(43) Pub. Date: Oct. 23, 2003

(54) Radioprotectants for radiopharmaceutical formulations

(76) Inventor: R. Keith Frank, Lake Jackson, TX (US)

Correspondence Address: THE DOW CHEMICAL COMPANY, INTELLECTUAL PROPERTY SECTION, P. O. BOX 1967, MIDLAND, MI, 48641-1967, US

(21) Appl. No.: 10/123,592

(22) Filed: Apr. 15, 2002

(51) Int. Cl. A61K 51/00

(52) U.S. Cl. 424/1.11

(57) ABSTRACT

A radiopharmaceutical composition comprising a radiopharmaceutical agent and salicylic acid or a salt thereof is disclosed. Also disclosed is a method of protecting a radiopharmaceutical agent comprising adding salicylic acid or a salt thereof to the radiopharmaceutical agent. The use of salicylic acid or a salt thereof as a radioprotecting agent provides for a high degree of protection against radiolysis while at the same time minimizing the impurities in the resulting radiopharmaceutical formulation.

### FIELD OF THE INVENTION

The present invention relates to radiation-protecting agents which protect the active ingredient of radiopharmaceutical formulations against premature decomposition through radiation.

### BACKGROUND OF THE INVENTION

Radiopharmaceuticals are drugs to be administered into a living body for the purpose of nuclear medicine diagnosis or radiotherapy. In general, radiopharmaceuticals comprise a radioisotope or an organic compound stably bound to a radioisotope as an active ingredient. Many radiopharmaceuticals are formulated as aqueous solutions. When an organic compound is used as an active ingredient, the radioisotope is incorporated into the molecular structure of active ingredient through covalent bond or coordination bond.

Diagnostic (or imaging) radiopharmaceuticals generally utilize gamma-emitting isotopes that can be detected from outside the body. Therapeutic radiopharmaceuticals generally utilize isotopes that emit particles, beta or alpha, or low energy gamma photons. Regardless of the radiation type (gamma, beta, or alpha) these radiations cause ionizations in matter, and are known collectively as ionizing radiation. The emitted particle or photon then interacts with water in the cell of a living being and forms hydroxyl radicals. These very reactive free radicals can then destroy tissue.

The same phenomenon of hydroxyl radical formation also occurs in a vial in which the radiopharmaceutical is packaged before it ever reaches the patient. These reactive hydroxyl radicals can interact with and destroy the organic portion of the radiopharmaceutical itself, rendering the radiopharmaceutical ineffective.

This radiolytic degradation, known as radiolysis, has been addressed by freezing or by using radioprotectants. Radioprotectants are molecules that typically work in a sacrificial mode to scavenge hydroxyl radicals. For example, the use of ascorbic acid is well known as a radioprotectant. U.S. Pat. No. 6,027,710 discloses other specific organic materials as radioprotecting agents. However, some of the organic materials disclosed are not as effective as others and can result in the formation of undesirable impurities. It would be desirable to provide a radio-protecting agent which resulted in higher efficacy and a better purity profile than those disclosed in the prior art.

### SUMMARY OF THE INVENTION

In one aspect, the present invention is a radiopharmaceutical composition comprising a radiopharmaceutical agent and salicylic acid or a salt thereof.

In a second aspect, the present invention is a method of protecting a radiopharmaceutical agent comprising: adding salicylic acid or a salt thereof to the radiopharmaceutical agent.

The use of salicylic acid or a salt thereof as a radioprotecting agent provides for a high degree of protection against radiolysis while at the same time minimizing the impurities in the resulting radiopharmaceutical formulation.

### BRIEF DESCRIPTION OF THE FIGURES

FIG. 1 is a chromatogram depicting the purity levels of an embodiment of the present invention after radiolytic degradation.

FIG. 2 is a chromatogram depicting purity levels of an embodiment of the prior art after radiolytic degradation.

### DETAILED DESCRIPTION OF THE INVENTION

As used herein, the term “salicylate” means salicylic acid or a salt thereof.

The radiopharmaceutical agent used in the present invention comprises an active ingredient radiolabeled with a radioisotope. As described above, the purpose for the addition of salicylate is to protect the active ingredient against radiolysis. Such protection is effective regardless of the type of radiation involved, namely, alpha, beta or gamma radiation. Accordingly, salicylate is effective for use with all kinds of radioisotopes used in radiopharmaceuticals. Such radioisotopes include ¹²⁵I, ¹³¹I, ²²⁵Ac, ²¹²Bi, ²¹³Bi, ²¹¹At, ¹⁵³Sm, ¹⁷⁷Lu, ¹⁵⁹Gd, ¹⁴⁹Pm, ¹⁴⁰La, ¹⁷⁵Yb, ¹⁶⁶Ho, 90Y, ⁴⁷Sc, ¹⁸⁶Re, ¹⁸⁸Re, ¹⁴²Pr, ^{99m}Tc, ⁶⁷Ga, ¹⁰⁵Rh, ⁹⁷Ru, ¹¹¹In, ^{113m}In, ^{115m}In.

The active ingredient for use in the present invention comprises a molecular structure containing the radioisotope. In some embodiments, the active ingredient may be a radioisotope complexed with a chelating agent such as those described in U.S. Pat. Nos. 5,435,990, 5,652,361 and 4,898,724, incorporated herein by reference. In other embodiments, the active ingredient may be a radiolabeled organic molecule as those described in WO 99/63547 and U.S. Pat. No. 6,315,979, incorporated herein by reference. Such active ingredients can be used for medical diagnosis or therapy.

Salicylate may be added to the radiopharmaceutical agent either before or after radiolabeling the active ingredient with the radioisotope. The form of the radiopharmaceutical agent may be freeze-dried, powder, mixture or liquid form.

Preferably, the concentration of salicylate in the final radiopharmaceutical formulation is from about 1-10% by weight based on weight of the final formulation. More preferably, the concentration of salicylate is from about 2-8% by weight and even more preferably from about 3-7% by weight. The temperature and pressure at which the salicylate acid is added to the radiopharmaceutical formulation are not critical.

The resulting radiopharmaceutical formulations include those suitable for parenteral (including subcutaneous, intramuscular, intraperitoneal, and intravenous), oral, rectal, topical, nasal, or ophthalmic administration. Formulations may be prepared by any methods well known in the art of pharmacy. Such methods include the step of bringing the radiopharmaceutical agent into association with a carrier, excipient or vehicle therefore. In general, the formulation may be prepared by uniformly and intimately bringing the radiopharmaceutical agent into association with a liquid carrier, a finely divided solid carrier, or both, and then, if necessary, shaping the product into desired formulation. In addition, the formulations of this invention may further include one or more accessory ingredient(s) selected from diluents, buffers, binders, disintegrants, surface active agents, thickeners, lubricants, preservatives, and the like. In addition, a treatment regime might include pretreatment with non-radioactive carrier.

Injectable formulations of the present invention may be either in suspensions or solution form. In the preparation of suitable formulations it will be recognized that, in general, the water solubility of the salt is greater than the acid form. In solution form the complex (or when desired the separate components) is dissolved in a physiologically acceptable carrier. Such carriers comprise a suitable solvent, preservatives, or buffers. Useful solvents include, for example, water, aqueous alcohols, glycols, and phosphonate or carbonate esters. Such aqueous solutions contain no more than 50 percent of the organic solvent by volume.

Injectable suspensions are compositions of the present invention that require a liquid suspending medium, with or without adjuvants, as a carrier. The suspending medium can be, for example, aqueous polyvinylpyrrolidone, inert oils such as vegetable oils or highly refined mineral oils, polyols, or aqueous carboxymethylcellulose. Suitable physiologically acceptable adjuvants, if necessary to keep the complex in suspension, may be chosen from among thickeners such as carboxymethylcellulose, polyvinylpyrrolidone, gelatin, and the alginates. Many surfactants are also useful as suspending agents, for example, lecithin, alkylphenol, polyethyleneoxide adducts, naphthalenesulfonates, alkylbenzenesulfonates, and polyoxyethylene sorbitane esters.

### EXAMPLES

The following examples are provided to further illustrate the present invention, and should not be construed as limiting thereof.

The materials used in the following examples are indicated below in Table 1.

TABLE 1
Sources of Materials

| Material | Source |
|---|---|
| Ascorbic acid, sodium salt, 99+% | Aldrich Chemical Co., Inc. |
| 2,5 dihydroxybenzoic acid, 98% | Aldrich Chemical Co., Inc. |
| Salicylic acid, sodium salt, 99+% | Aldrich Chemical Co., Inc. |
| Dextran, avg. mw 9300 | Sigma Chemical Co. |
| Ferrous sulfate (· 7 H₂O), AR | Mallinckrodt Chemical Works |
| 1,10-Phenanthroline (· H₂O), 99+% | Aldrich Chemical Co., Inc. |
| Glutathione, reduced, 98% | Aldrich Chemical Co., Inc. |
| Sodium hydroxide, 50% | Fisher Scientific |
| Sodium carbonate | Aldrich Chemical Co., Inc. |
| Water, deionized | in-house Barnstead NANOpure |

### Example 1 and Comparative Examples 2-14

I-HBS (sodium 3-(¹²⁵I)iodo-4-hydroxybenzenesulfonate) solutions were prepared, as in U.S. Pat. No. 6,315,979, using various potential radioprotectants as shown below in Table 2. Conical glass vials (2 mL, Kimble Glass, Inc.) were prepared containing pre-weighed amounts of the solid potential radioprotectants to be evaluated. I-HBS (1 mL, 195 mCi/mL) was then dispensed into each vial.

TABLE 2
Identification and Contents of Vials

| Vial | Description | Contents |
|---|---|---|
| 1 | Salicylate | 50 mg sodium salicylate |
| Comp 2 | Control | Empty |
| Comp 3 | Ascorbate | 50 mg sodium ascorbate |
| Comp 4 | Ascorbate | 75 mg sodium ascorbate |
| | 7.5% | |
| Comp 5 | DHB | 50 mg sodium 2,5-dihydroxybenzoate |
| Comp 6 | Dextran | 50 mg dextran (mw ˜9300) |
| Comp 7 | FePhen | 50 mg iron (II) tris-1,10- |
| | | phenanthroline sulfate |
| Comp 8 | FeDTPA | 50 mg iron (II) DTPA |
| Comp 9 | FeBisIDA | 50 mg iron (II) Bis-IDA |
| Comp 10 | FeSalicylate | 50 mg iron (II) bis-salicylate |
| Comp 11 | FeDOTA | 50 mg iron (II) DOTMP |
| Comp 12 | Glutathione | 50 mg glutathione (reduced) |
| Comp 13 | Asc/FePhen | 25 mg each |
| Comp 14 | Asc/Dextran | 25 mg each |

Analytical samples were taken from each vial on days 0,2,8,15,23 and 30. The samples were analyzed by HPLC for free ¹²⁵I iodide, which is the major radiolytic impurity due to radiolysis. The results from these analyses, expressed as percent free iodide (¹²⁵I), are listed below in Table 3. These numbers represent the percent of the radioactivity (area percent) associated with the ¹²⁵I retention time in the radiometric chromatogram.

TABLE 3
Results of HPLC Analysis (% Free ¹²⁵Iodide)

| | | | | | | | Comp | | | | | | | |
|---|---|---|---|---|---|---|---|---|---|---|---|---|---|---|
| | | | Comp | Comp | | | 7 | Comp | Comp | Comp | Comp | Comp | Comp | Comp |
| | 1 | Comp | 3 | 4 | Comp | Comp | Fe- | 8 | 9 | 10 | 11 | 12 | 13 | 14 |
| Times | Salicy- | 2 | Ascorbate | Ascorbate | 5 | 6 | Phenan- | Fe- | Fe-Bis- | Fe- | Fe- | Gluta- | Asc/ | Asc/ |
| (Days) | late | Control | (5%) | (7.5%) | DHB | Dextran | throline | DTPA | IDA | Salicylate | DOTA | thione | FePhen | Dex |
| 0 | 0.00 | 0.00 | 0.96 | 0.50 | 0.15 | 0.00 | 0.00 | 0.37 | 0.10 | 0.09 | 0.34 | 1.30 | 0.25 | 0.35 |
| 2 | 1.02 | 6.01 | 3.77 | 4.16 | 0.51 | 7.41 | 0.74 | 3.08 | 1.16 | 1.56 | 4.49 | 6.76 | 1.56 | 4.18 |
| 8 | 0.57 | 20.43 | 13.15 | 12.35 | 0.00 | 14.15 | 0.82 | 14.16 | 1.72 | 3.11 | 15.07 | 10.29 | 2.56 | 18.08 |
| 15 | 3.70 | 43.49 | 27.72 | 24.27 | 3.02 | 41.23 | 3.01 | 29.50 | 6.95 | 11.13 | 37.11 | 15.25 | 5.47 | 33.49 |
| 23 | 3.85 | 43.54 | 35.04 | 31.82 | 3.66 | 53.49 | 3.40 | 40.05 | 12.66 | 11.85 | 47.99 | 20.04 | 7.59 | 44.51 |
| 30 | 8.16 | 42.98 | 43.09 | 39.19 | 8.55 | 72.60 | 3.48 | 49.83 | 20.87 | 30.03 | 61.13 | 26.85 | 8.73 | 54.03 |

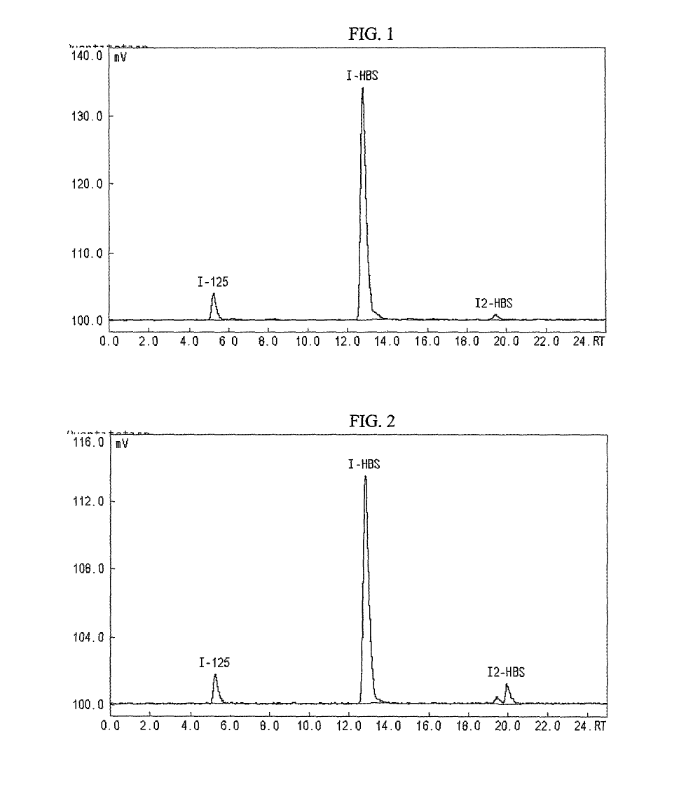

Surprisingly, the use of salicylate (Example 1) results in fewer impurities than the radioprotecting agents disclosed in the prior art. FIG. 1 is the chromatogram showing the purity profile for salicylate (Example 1). Peaks are shown for ¹²⁵I, I-HBS and I₂-HBS (sodium 3,5-(¹²⁵I)bisiodo-4-hydroxybenzenesulfonate), as identified in FIG. 1. FIG. 2 is a chromatogram depicting the purity profile for dihydroxybenzoic acid (Comparative Example 5). Again, peaks are shown for ¹²⁵I, I-HBS and I₂-HBS. However, there is an additional peak with a retention time of approximately 19.4 minutes, depicting unidentified impurities which are present in the resulting formulation. Such a peak does not exist in FIG. 1.

What is claimed is:

1. A radiopharmaceutical composition comprising a radiopharmaceutical agent and salicylic acid or a salt thereof.

2. The composition according to claim 1 wherein the radiopharmaceutical agent comprises a radiolabeled active ingredient labeled with ¹²⁵I, ¹³¹I, ²²⁵Ac, ²¹²Bi, ²¹³Bi, ²¹¹At, ¹⁵³Sm, ¹⁷⁷Lu, ¹⁵⁹Gd, ¹⁴⁹Pm, ¹⁴⁰La, ¹⁷⁵Yb, ¹⁶⁶Ho, ⁹⁰Y, ⁴⁷Sc, ¹⁸⁶Re, ¹⁸⁸Re, ¹⁴²Pr, ^{99m}Tc, ⁶⁷Ga, ¹⁰⁵Rh, ⁹⁷Ru, ¹¹¹In, ^{113m}In, or ^{115m}In.

3. The composition according to claim 2 wherein the radiopharmaceutical agent comprises a radiolabeled active ingredient labeled with ¹²⁵I.

4. The composition according to claim 2 wherein the active ingredient comprises sodium 3-(¹²⁵I)iodo-4-hydroxybenzenesulfonate.

5. The composition according to claim 1 wherein the concentration of salicylate in the radiopharmaceutical formulation is between 1.0 and 10.0 weight percent.

6. A method of protecting a radiopharmaceutical agent comprising: adding salicylic acid or a salt thereof to a radiopharmaceutical agent.

7. The method according to claim 6 wherein the radiopharmaceutical agent comprises a radiolabeled active ingredient labeled with ¹²⁵I, ¹³¹I, ²²⁵Ac, ²¹²Bi, ²¹³Bi, ²¹¹At, ¹⁵³Sm, ¹⁷⁷Lu, ¹⁵⁹Gd, ¹⁴⁹Pm, ¹⁴⁰La, ¹⁷⁵Yb, ¹⁶⁶HO, ⁹⁰Y, ⁴⁷Sc, ¹⁸⁶Re, ¹⁸⁸Re, ¹⁴²Pr, ^{99m}Tc, ⁶⁷Ga, ¹⁰⁵Rh, ⁹⁷Ru, ¹¹¹In, ^{113m}In, or ^{115m}In.

8. The method according to claim 7 wherein the radiopharmaceutical agent comprises a radiolabeled active ingredient labeled with ¹²⁵I.

9. The method according to claim 7 wherein the active ingredient comprises sodium 3-(¹²⁵I)iodo-4-hydroxybenzenesulfonate.

10. The method according to claim 6 wherein the salicylic acid or salt thereof is added at a concentration between 1.0 and 10.0 percent by weight.
